(19) United States
(12) Patent Application Publication
(10) Pub. No.: US 2007/0025935 A1
(43) Pub. Date: Feb. 1, 2007

(54) Nail varnish containing a polyoxyethylene/polyoxypropylene block polymer

(75) Inventor: Philippe Ilekti, Maison-Alfort (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/495,729

(22) Filed: Jul. 31, 2006

Related U.S. Application Data
(60) Provisional application No. 60/708,403, filed on Aug. 16, 2005.

(30) Foreign Application Priority Data
Jul. 29, 2005 (FR) 05 08179

(51) Int. Cl. A61K 8/86

(52) U.S. Cl. 424/61

(57) ABSTRACT

Nail varnish composition containing, in a cosmetically acceptable medium comprising an organic solvent medium, at least one block copolymer of formula (I) and at least one gelling agent. The invention also relates to a nail varnish composition containing at least one copolymer comprising at least one polyethylene glycol block and at least one polypropylene glycol block and at least one gelling agent. The invention further relates to the use of such compositions to obtain a glossy film with good staying power when deposited on the nails.

### REFERENCE TO PRIOR APPLICATIONS

This application claims priority to U.S. provisional application 60/708,403 filed Aug. 16, 2005, and to French patent application 0508179 filed Jul. 29, 2005, both incorporated herein by reference.

### FIELD OF THE INVENTION

The present invention relates to a nail varnish comprising a polyoxy-alkylene block copolymer. The invention also relates to a nail makeup or care process.

The nail varnish composition of the invention may be used as a varnish base or “base coat”, as a nail makeup product, as a finishing composition, also known as a “top coat”, to be applied over the nail makeup product, as a cosmetic nailcare product, or as any combination thereof. These compositions may be applied to human nails or to false nails.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Nail varnish compositions generally comprise solid particles such as pigments, nacres and fillers, which are in dispersion in the aqueous continuous medium or the organic solvent medium of the composition.

However, these particles have a tendency to sediment out over time, due to their density which is greater than that of the continuous medium in which they are dispersed. This sedimentation is reflected by a change in the macroscopic appearance of the composition, and in particular, in the case of coloured nail varnishes, by heterogeneity of the colour of the varnish and a deterioration of the staying power of the varnish film on the nails.

In order to improve the stabilization of compositions, formulators can use thickeners or gelling agents, for instance bentone; however, the incorporation of these agents in an amount required for stabilization of the varnish has a tendency to make the varnish film matt, which is not desirable in the case of nail varnishes, for which it is desired to obtain a glossy film on the nail.

### SUMMARY OF THE INVENTION

The inventor has now discovered, surprisingly, that the incorporation into a nail varnish with an organic solvent medium of a particular polyoxyalkylene block copolymer provides a nail varnish composition that shows good stability and good colour homogeneity over time, and also the formation of a glossy film on the nails that shows good staying power over time.

### DETAILED DESCRIPTION OF THE PREFERRED EMBODIMENTS

One subject of the invention is a nail varnish composition comprising, in a cosmetically acceptable medium comprising an organic solvent medium:
at least one block copolymer of formula (I):
   HO—(CH2—CH2—O)n-(CxH2xO)m-(CH2—CH2—O)p—H,
   in which n, m and p are, independently, integers ranging from 1 to 1000 and x is an integer strictly greater than 2 and less than or equal to 10, where said polymer is present in an amount greater than or equal to 0.1% by weight relative to the total weight of the composition and
at least one gelling agent.

Another subject of the invention is a nail varnish composition comprising, in a cosmetically acceptable medium comprising an organic solvent medium, at least one copolymer comprising at least one polyethylene glycol block and at least one polypropylene glycol block, the copolymer having a molecular weight of greater than or equal to 1000 g/mol and said polymer is present in an amount greater than or equal to 0.1% by weight relative to the total weight of the composition, and at least one gelling agent.

These particular polyoxyalkylene block copolymers allow the composition according to the invention to be stabilized and may be formulated in supplement to a standard gelling agent, for instance bentone or silica, without having the drawback of adding large amounts of bentone, which leads to a loss of gloss of the varnish film.

For the purposes of the invention, the term “cosmetically acceptable medium” means a non-toxic medium that may be applied to human facial skin, integuments or lips.

Another subject of the invention is also a non-therapeutic cosmetic process for making up or caring for the nails, comprising the application to the nails of at least one coat of a nail varnish composition as described herein.

A subject of the invention is also the use, in a nail varnish composition with an organic solvent medium, of at least one block copolymer of formula (I) below:
HO—(CH2—CH2—O)n-(CxH2xO)m-(CH2—CH2—O)p—H
in which n, m and p are independently integers ranging from 1 to 1000 and x is an integer greater than 2 and less than or equal to 10,
to obtain a nail varnish that shows good stability and good colour homogeneity over time, and also the formation of a glossy film with good staying power on the nails.

A subject of the invention is also the use, in a nail varnish composition with an organic solvent medium, of at least one copolymer comprising at least one polyethylene glycol block and at least one polypropylene glycol block, the copolymer having a molecular weight of greater than or equal to 1000 g/mol, to obtain a nail varnish that shows good stability and good colour homogeneity over time, and also the formation of a glossy film on the nails that shows good staying power.

Block Copolymer

The term “block copolymer” means a polymer comprising at least 2 different blocks and preferably at least 3 different blocks.

A preferred block copolymer of the composition according to the invention comprises polyoxyalkylenated blocks and corresponds in particular to formula (I) below:
HO—(CH2—CH2—O)n-(CxH2xO)m-(CH2—CH2—O)p—H
in which n, m and p are, independently, integers ranging from 1 to 1000 and x is an integer strictly greater than 2 and less than or equal to 10.

According to another embodiment, the block copolymer comprises at least one polyethylene glycol block and at least one polypropylene glycol block. For example, it may then correspond to the following formula:
HO—(CH2—CH2—O)n-(CxH2xO)m—H
in which n and m are integers between 1 and 1000 and x is an integer strictly greater than 2 and less than 10.

The block copolymer of the composition advantageously has a molecular weight of greater than or equal to 1000 g/mol, preferably greater than or equal to 1500 g/mol and better still greater than 2000 g/mol.

As copolymers that may be used in the composition according to the invention, mention may be made especially of copolymers of propylene oxide and of ethylene oxide, also known as EO/PO polycondensates, for instance the polyethylene glycol/polypropylene glycol/polyethylene glycol triblock polycondensates sold under the name Synperonic, for instance Synperonic PE/L44, Synperonic PE/L64 (13EO/13PO/13EO) of MW 2900 and Synperonic PE/F127 by the company ICI, and mixtures thereof.

The block copolymer(s) are preferably present in an amount greater than or equal to 0.1% by weight, relative to the total weight of the composition, preferably greater than or equal to 0.5%, better still greater than or equal to 1% by weight

These block copolymer(s) may be present in any amount, including for example an amount ranging from 0.1% to 15% by weight, preferably from 0.5% to 10% and better still from 1% to 8% by weight relative to the total weight of the composition.

Organic Solvent Medium

The cosmetically acceptable medium of the cosmetic composition according to the invention comprises an organic solvent medium comprising an organic solvent or a mixture of organic solvents.

The organic solvent may for example be chosen from:
ketones that are liquid at room temperature, such as methyl ethyl ketone, methyl isobutyl ketone, diiso-butyl ketone, isophorone, cyclohexanone or acetone;
alcohols that are liquid at room temperature, such as ethanol, isopropanol, diacetone alcohol, 2-butoxyethanol or cyclohexanol;
propylene glycol ethers that are liquid at room temperature, such as propylene glycol monomethyl ether, propylene glycol monomethyl ether acetate or dipropylene glycol mono-n-butyl ether;
cyclic ethers such as γ-butyrolactone;
short-chain esters (containing from 3 to 8 carbon atoms in total) such as ethyl acetate, methyl acetate, propyl acetate, isopropyl acetate, n-butyl acetate, isopentyl acetate, methoxypropyl acetate or butyl lactate;
ethers that are liquid at room temperature, such as diethyl ether, dimethyl ether or dichlorodiethyl ether;
alkanes that are liquid at room temperature, such as decane, heptane, dodecane or cyclohexane;
alkyl sulfoxides such as dimethyl sulfoxide;
aldehydes that are liquid at room temperature, such as benzaldehyde or acetaldehyde;
ethyl 3-ethoxypropionate;
carbonates such as propylene carbonate or dimethyl carbonate;
acetals such as methylal;
and mixtures thereof.

Preferably, the solvent is chosen from short-chain esters containing from 3 to 8 carbon atoms in total, such as ethyl acetate, methyl acetate, propyl acetate, isopropyl acetate, n-butyl acetate, isopentyl acetate, methoxypropyl acetate or butyl lactate, and mixtures thereof.

The organic solvent medium may be present in any amount, and for example may represent from 10% to 95% by weight, preferably from 15% to 80% by weight and better still from 20% to 60% by weight relative to the total weight of the composition.

The composition according to the invention may optionally comprise an aqueous medium: in this case, the aqueous medium may be present in any amount, such as in a content of less than or equal to 2% by weight and preferably less than or equal to 1% by weight relative to the total weight of the composition.

Gelling Agent

Useful gelling agents herein include, for instance, silica and modified clays such as bentones. Other useful gelling agents include other modified clays, for instance, hectorites modified with an ammonium chloride of a C10 to C22-fatty acid, for instance hectorite modified with distearyldimethylammonium chloride; fumed silica optionally hydrophobically surface-treated, with a particle size of less than 1 μm; galactomannans comprising from one to six and better from two to four hydroxyl groups per saccharide, substituted with a saturated or unsaturated alkyl chain, for instance guar gum alkylated with C1 to C6 and better still C1 to C3 alkyl chains, and more particularly ethylated guar having a degree of substitution of 2 to 3, such as the product sold by the company Aqualon under the name N-Hance-AG; ethylcellulose, for instance the products sold under the name Ethocel by Dow Chemical; gums, especially silicone gums, for instance PDMSs having a viscosity>100 000 centistokes.

Preferred gelling agents herein include silicas, clays such as montmorillonite, modified clays such as bentones, and polysaccharide alkyl ethers, and mixtures thereof. Also preferred are silicas, especially hydrophobic silicas, such as those described in document EP-A-898 960 and sold, for example, under the references “Aerosil R812®” by the company Degussa, “Cab-O-Sil TS-530®”, “Cab-O-Sil TS-610®”, “Cab-O-Sil TS-720®” by the company Cabot, “Aerosil R972®” and “Aerosil R974®” by the company Degussa; clays such as montmorillonite, modified clays such as bentones, for example stearalkonium hectorite and stearalkonium bentonite, polysaccharide alkyl ethers (especially in which the alkyl group contains from 1 to 24, preferably from 1 to 10, better still from 1 to 6 and more especially from 1 to 3 carbon atoms) such as those described in document EP-A-898 958.

According to one particular embodiment, the composition comprises a thickener chosen from bentones, in a content of less than or equal to 2% by weight relative to the total weight of the composition.

The gelling agent may be present in any amount, preferably is present in an amount providing the desired viscosity, and for example may represent from 0.01% to 50% by weight, preferably from 0.1% to 15% by weight and better still from 0.1% to 5% by weight relative to the total weight of the composition. According to a specific embodiment, the total proportion of gelling agent(s) in the composition is less than 5% by weight relative to the total weight of the composition, preferably less than 3% by weight.

Film-Forming Polymer

The composition may advantageously comprise a film-forming polymer.

According to the present invention, the term “film-forming polymer” means a polymer capable, by itself or in the presence of an auxiliary film-forming agent, of forming a continuous and adherent film on a support and especially on keratin materials.

Among the film-forming polymers that may be used in the composition of the present invention, mention may be made of synthetic polymers, of free-radical type or of polycondensate type, and polymers of natural origin, and mixtures thereof.

The film-forming polymer may be chosen in particular from cellulose-based polymers such as nitrocellulose, cellulose acetate, cellulose acetobutyrate, cellulose acetopropionate or ethyl cellulose, or polyurethanes, acrylic polymers, vinyl polymers, polyvinyl butyrals, alkyd resins, resins derived from aldehyde condensation products such as arylsulfonamide formaldehyde resins, for instance toluenesulfonamide formaldehyde resin, arylsulfonamide epoxy resins or ethyl tosylamide resins.

Film-forming polymers that may especially be used include nitrocellulose RS ⅛sec; RS ¼sec.; ½sec.; RS 5 sec.; RS 15 sec.; RS 35 sec.; RS 75 sec.; RS 150 sec; AS ¼sec.; AS ½sec.; SS ¼sec.; SS ½sec.; SS 5 sec., sold especially by the company Hercules; the toluenesulfonamide formaldehyde resins “Ketjentflex MS80” from the company Akzo or “Santolite MHP” and “Santolite MS 80” from the company Faconnier or “Resimpol 80” from the company Pan Americana, the alkyd resin “Beckosol ODE 230-70-E” from the company Dainippon, the acrylic resin “Acryloid B66” from the company Rohm & Haas, and the polyurethane resin “Trixene PR 4127” from the company Baxenden.

According to one embodiment of the invention, the film-forming polymer is a film-forming linear block ethylenic polymer, which preferably comprises at least one first block and at least one second block with different glass transition temperatures (Tg), the first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in documents EP 1 411 069 or WO 04/028 488.

The additional film-forming polymer may be present in the composition according to the invention in any amount, including in a content ranging from 0.1% to 60% by weight, preferably ranging from 2% to 40% by weight and better still from 5% to 25% by weight relative to the total weight of the composition.

Auxiliary Film-Forming Agent

To improve the film-forming properties of the nail varnish composition, an auxiliary film-forming agent may be provided.

Such an auxiliary film-forming agent may be chosen from any compound known to those skilled in the art as being capable of fulfilling the desired function, and may be chosen especially from plasticizers and coalescers for the film-forming polymer(s).

Thus, the composition may also comprise at least one plasticizer and/or one coalescer. In particular, mention may be made, alone or as a mixture, of common plasticizers and coalescers, such as:
glycols and derivatives thereof, such as diethylene glycol ethyl ether, diethylene glycol methyl ether, diethylene glycol butyl ether or diethylene glycol hexyl ether, ethylene glycol ethyl ether, ethylene glycol butyl ether or ethylene glycol hexyl ether;
polyethylene glycols, polypropylene glycols, polyethylene glycol-polypropylene glycol copolymers and mixtures thereof, especially polypropylene glycols of high molecular weight having, for example, a molecular mass ranging from 500 to 15 000, for instance:
glycol esters;
propylene glycol derivatives and in particular propylene glycol phenyl ether, propylene glycol diacetate, dipropylene glycol ethyl ether, tripropylene glycol methyl ether, diethylene glycol methyl ether and propylene glycol butyl ether;
acid esters, especially carboxylic acid esters, such as citrates, phthalates, adipates, carbonates, tartrates, phosphates and sebacates;
esters derived from the reaction of a monocarboxylic acid of formula R₁₁COOH with a diol of formula HOR₁₂OH with R₁₁ and R₁₂, which may be identical or different, representing a linear, branched or cyclic, saturated or unsaturated hydrocarbon-based chain preferably containing from 3 to 15 carbon atoms and optionally containing one or more heteroatoms such as N, C or S, in particular the monoester resulting from the reaction of isobutyric acid and octanediol such as 2,2,4-trimethyl-1,3-pentanediol, such as the product sold under the name Texanol Ester Alcohol by the company Eastman Chemical;
oxyethylenated derivatives such as oxyethylenated oils, especially plant oils such as castor oil;
dimethicone copolyols, especially containing α, ω-propyl polyoxypropylene groups; and
mixtures thereof.
   Dyestuff

The composition according to the invention may also comprise one or more dyestuffs chosen from water-soluble dyes and pulverulent dyestuffs, for instance pigments, nacres and flakes that are well known to those skilled in the art. The dyestuffs may be present in the composition in any amount, such as in a content ranging from 0.01% to 50% by weight and preferably from 0.01% to 30% by weight relative to the weight of the composition.

The term “pigments” should be understood as meaning white or coloured, mineral or organic particles of any form, which are insoluble in the physiological medium, and which are intended to colour the composition.

The term “nacres” should be understood as meaning iridescent particles of any form, especially produced by certain molluscs in their shell or else synthesized.

The pigments may be white or coloured, and mineral and/or organic. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, for instance aluminium powder or copper powder.

Among the organic pigments that may be mentioned are carbon black, pigments of D&C type and lakes based on cochineal carmine or on barium, strontium, calcium or aluminium.

The nacreous pigments may be chosen from white nacreous pigments such as mica coated with titanium or with bismuth oxychloride, coloured nacreous pigments such as titanium mica coated with iron oxides, titanium mica coated especially with ferric blue or with chromium oxide, titanium mica coated with an organic pigment of the abovementioned type and also nacreous pigments based on bismuth oxychloride.

The water-soluble dyes are, for example, beetroot juice and methylene blue.

The composition according to the invention may additionally comprise one or more fillers, especially in a content ranging from 0.01% to 50% by weight and preferably ranging from 0.01% to 30% by weight, relative to the total weight of the composition. The term “fillers” should be understood as meaning colour-less or white, mineral or synthetic particles of any shape, which are insoluble in the medium of the composition, irrespective of the temperature at which the composition is manufactured. These fillers serve especially to modify the rheology or the texture of the composition.

The fillers may be mineral or organic in any form, platelet-shaped, spherical or oblong, irrespective of the crystallographic form (for example leaflet, cubic, hexagonal, orthorhombic, etc.). Mention may be made of talc, mica, silica, kaolin, polyamide (Nylon®) powder (Orgasol® from Atochem), poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie) or acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids containing from 8 to 22 carbon atoms and preferably from 12 to 18 carbon atoms, for example zinc, magnesium or lithium stearate, zinc laurate or magnesium myristate.

Other Additives

The composition may also comprise other ingredients such as those commonly used in cosmetic compositions. Such ingredients may be chosen for example from spreading agents, wetting agents, dispersants, antifoams, preserving agents, UV stabilizers, active agents, surfactants, moisturizers, fragrances, neutralizers, stabilizers and antioxidants.

Needless to say, a person skilled in the art will take care to select this or these optional additional compound(s), and/or the amount thereof, such that the advantageous properties of the composition for the use according to the invention are not, or are not substantially, adversely affected by the envisaged addition.

According to another aspect, a subject of the invention is a nail varnish product comprising: i) a container delimiting at least one compartment, the container being closed by means of a closing member, and ii) a composition according to the invention placed inside the compartment.

The container may be in any suitable form. It may especially be in the form of a bottle and may be at least partly made of a material such as glass. However, materials other than glass may be used, for instance thermoplastics such as PP or PE, or a metal.

The closing member may be coupled to the compartment by screwing in the closed position of the container. Alternatively, the coupling between the closing member and the container may take place other than by screwing, especially by click-fastening.

The container is preferably equipped with an applicator, which may be in the form of a brush consisting of at least one tuft of bristles. Alternatively, the applicator is in a form other than a brush, for example in the form of a spatula or a foam tip.

The examples that follow illustrate the invention in a non-limiting manner. Unless otherwise indicated, the amounts indicated are mass percentages.

### EXAMPLE 1

### Nail Varnish

| | |
|---|---|
| Nitrocellulose containing 30% isopropyl alcohol | 14 |
| (viscosity: E22-1/2 S) | |
| Nitrocellulose containing 30% isopropyl alcohol | 2.45 |
| (Azur E80 from Bergerac) | |
| Glycerophthalic alkyd resin esterified with branched | 14 |
| fatty acids⁽¹⁾ at 70% in ethyl acetate | |
| Tributyl acetyl citrate | 2.45 |
| N-Ethyl-O,P-toluenesulfonamide | 3.42 |
| Stearylbenzyldimethylammonium-modified hectorite | 0.7 |
| Titanium oxide coated with polyethylene oxide (97/3) | 0.047 |
| Lake Red 34 | 0.26 |
| Ethylene oxide/propylene oxide/ethylene oxide condensate | 2.45 |
| (13EO/30PO/13EO)⁽²⁾ | |
| Isopropyl alcohol | 3.23 |
| Citric acid monohydrate | 0.03 |
| Ethyl acetate | 20 |
| Butyl acetate | qs 100 |

⁽¹⁾sold by Shell under the brand name Cardura 30 ®.
⁽²⁾sold by Uniqema under the reference Synperonic PE/L64

The gloss of the varnish was measured according to the following protocol:

A coat with a wet thickness of 300 μm of the composition whose mean gloss it is sought to be evaluated is spread onto a Leneta brand contrast card of reference Form 1A Penopac, using an automatic spreader. The coat covers the white background and the black background of the card. The composition is left to dry for 24 hours on a bench thermostatically maintained at 30° C., and the gloss at 20° and 60° is then measured on the white background (3 measurements) and on the black background (3 measurements) using a Byk Gardner brand glossmeter of reference microTri-Gloss.

The nail varnish of Example 1 has a gloss, measured at 20°, of 74 and a gloss, measured at 60°, of 89.

The above written description of the invention provides a manner and process of making and using it such that any person skilled in this art is enabled to make and use the same, this enablement being provided in particular for the subject matter of the appended claims, which make up a part of the original description and including a nail varnish composition comprising, in a cosmetically acceptable medium comprising:
at least one block copolymer of formula (I) below:
   HO—(CH2—CH2—O)n-(CxH2xO)m-(CH2—CH2—O)p—H,
   in which n, m and p are, independently, integers ranging from 1 to 1000 and x is an integer strictly greater than 2 and less than or equal to 10, where said copolymer is present in an amount greater than or equal to 0.1% by weight relative to the total weight of the composition and
at least one gelling agent.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A nail varnish composition comprising, in a cosmetically acceptable medium comprising an organic solvent medium:
at least one block copolymer of formula (I):
HO—(CH2—CH2—O)n-(CxH2xO)m-(CH2—CH2—O)p—H,
in which n, m and p are, independently, integers ranging from 1 to 1000 and x is an integer greater than 2 and less than or equal to 10, where said copolymer is present in an amount greater than or equal to 0.1% by weight relative to the total weight of the composition, and
at least one gelling agent.

2. A nail varnish composition comprising, in a cosmetically acceptable medium comprising an organic solvent medium, at least one gelling agent, and at least one copolymer comprising at least one polyethylene glycol block and at least one polypropylene glycol block, the copolymer having a molecular weight of greater than or equal to 1000 g/mol and the copolymer being present in an amount greater than or equal to 0.1% by weight relative to the total weight of the composition.

3. The composition according to claim 2, wherein the polymer corresponds to the following formula:
HO—(CH2—CH2—O)n-(CxH2xO)m—H
in which n and m are integers between 1 and 1000 and x is an integer greater than 2 and less than or equal to 10.

4. The composition according to claim 1, wherein the block copolymer is present in an amount ranging from 0.1% to 15% by weight relative to the total weight of the composition.

5. The composition according to claim 1, wherein the organic solvent medium comprises at least one organic solvent chosen from:
ketones that are liquid at room temperature,
alcohols that are liquid at room temperature;
propylene glycol ethers that are liquid at room temperature;
cyclic ethers;
short-chain esters containing from 3 to 8 carbon atoms in total;
ethers that are liquid at room temperature;
alkanes that are liquid at room temperature;
alkyl sulfoxides;
aldehydes that are liquid at room temperature;
ethyl 3-ethoxypropionate;
carbonates;
acetals;
and mixtures thereof.

6. The composition according to claim 1, wherein the organic solvent medium represents from 10% to 95% by weight relative to the total weight of the composition.

7. The composition according to claim 1, wherein the gelling agent is chosen from silicas, clays, modified clays, polysaccharide alkyl ethers, and mixtures thereof.

8. The composition according to claim 1, wherein the gelling agent is present in a content ranging from 0.01% to 15% by weight relative to the total weight of the composition.

9. The composition according to claim 1, wherein the total proportion of gelling agent(s) in the composition is less than 5% by weight relative to the total weight of the composition.

10. The composition according to claim 1, wherein it comprises a positive amount of bentone in a content of less than or equal to 2% by weight relative to the total weight of the composition.

11. The composition according to claim 1, further comprising a film-forming polymer.

12. The composition according to claim 11, wherein the film-forming polymer is present in a content ranging from 0.1% to 60% by weight relative to the total weight of the composition.

13. The composition according to claim 1, wherein it further comprises a dyestuff.

14. The composition according to claim 13, wherein the dyestuff is present in a content ranging from 0.01% to 50% by weight relative to the weight of the composition.

15. A method, comprising the application to the nails of at least one coat of a nail varnish composition according to claim 1.

16. A cosmetic assembly comprising:
i) a closable container defining at least one compartment, and
ii) a composition according to claim 1 located inside the compartment.

17. The cosmetic assembly according to claim 16, wherein the container is at least partly made of glass.

18. The cosmetic assembly according to claim 16, wherein it further comprises an applicator in the form of a brush comprising at least one tuft of bristles.

19. The cosmetic assembly according to claim 16, wherein it further comprises an applicator other than a brush, such as a spatula or a foam tip.

20. The cosmetic assembly according to claim 19, wherein said applicator other than a brush is a spatula or a foam tip.
